(19) United States
(12) Patent Application Publication
(10) Pub. No.: US 2008/0095802 A1
(43) Pub. Date: Apr. 24, 2008

(54) Targeted delivery of cytotoxic drugs A) to activated lymphocytes in patients iwth transplanted organs, B) as radiosensitizers to cancer cells in paients undergoing radiation therapy, and C) in vitaminpbinding proteins as drug carriers in the diagnosis and treatment of cancer

(76) Inventor: Ward Page Faulk, St. Simons Islands, GA (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/952,181

(22) Filed: Dec. 7, 2007

Related U.S. Application Data
(63) Continuation of application No. 10/311,302, filed on Dec. 26, 2002, now abandoned.
(60) Provisional application No. 60/214,389, filed on Jun. 28, 2000.

(51) Int. Cl. A61K 35/74; A61K 31/337; A61K 31/517; A61K 38/13; A61P 37/00; A61K 38/40; A61K 38/00; A61K 31/704

(52) U.S. Cl. 424/236.1; 514/11; 514/2; 514/249; 514/34; 514/449; 514/8

(57) ABSTRACT

This invention relates to the targeted delivery of cytotoxic drugs A) to lymphocytes responsible for the rejection of transplanted tissues, such as kidneys or hearts or bone marrow cells; B) the use of a non-toxic naturally existing delivery system to transport high concentrations of radiosensitizers to cancer cells; and C) to the targeted delivery of drugs in medical diagnosis and treatment of cancer.

This application is a continuation of Ser. No. 10/311,302, filed Dec. 26, 2006, which is a 35 U.S.C. 371 National Phase Entry Application from PCT/US01/20444, filed Jun. 28, 2001, which claims the benefit of U.S. Provisional 60/214,389 filed Jun. 28, 2000, the disclosure of which is incorporated herein in its entirety by reference.

### FIELD OF THE INVENTION

This invention relates to A) the targeted delivery of cytotoxic drugs to lymphocytes responsible for the rejection of transplanted issues, such as bone marrow, kidneys or hearts; B) the use of a non-toxic naturally existing delivery system to transport high concentrations of radiosensitizers to cancer cells; and C) to the targeted delivery of drugs in medical diagnosis and treatment of cancer.

### BACKGROUND OF THE INVENTION

A) Transplantation—The immunological system is designed to contain a population of lymphocytes that function as immunosurveillance cells (Cohn, Theoretical Med & Bioethics 1998; 19: 475). Those cells serve as police that cruise around the body looking for foreign antigens, such as bacteria, viruses or non-self antigens.

Thousands of years ago the policing cells posed a problem in mammalian pregnancy. Certain cells in mammal embryos contact maternal blood, and these were rejected as non-self antigens. This problem was solved by the development of specialized forms of tolerance, such as clonal deletion, clonal anergy and apoptosis or programmed cell death (Jiang & Vacchio, J Immunol 1998; 160: 3086), thus allowing mammals to proliferate through sexual reproduction.

However, the immunosurveillance issue again became a problem with the introduction of transplantation into medicine (Starzl & Demetris, Theoretical Med & Bioethics 1998; 19: 441). Unlike pregnancy, this problem was not solved by adapting immunological mechanisms of tolerance. Instead, a new pharmaceutical era of immunosuppression drugs developed to combat the rejection responses mounted by organ transplant recipients.

Today it still is not possible to manipulate or engineer the immunological system to accept grafts of non-self tissues, so ever more powerful and toxic immunosuppression drugs are being produced. In 1999 alone, the USA market for immunosuppression drugs amounted to sales of slightly more than one billion dollars (data from MIS Health-MIDAS). In fact, it must be said that transplantation results as judged by organ survival times have continued to improve as a result of immunosuppression therapy, but such treatment is a delicate balance between rejection due to under-treatment and infection or malignancies due to over-treatment (Bergan et al., Tids Den Norske Laegefosen 1999; 119: 3615).

Lymphomas are the most common tumors in immunosuppressed transplant recipients, and the incidence can be as high as 30% in recipients who are setonegative for EBV (Epstein Barr Virus) before transplantation (Swinnen, Annals Oncol 2000; 11 (suppl 1): 45). Cytomegalovirus infections also are common in kidney transplant recipients, reaching in some studies a cumulative infection rate of 38% at 6 months after transplantation (Flechner et al., Transplant Proc 1999; 31: 1255). Although the mortality rate from infections has decreased, it remains a major cause of death that includes fungi and protozoa, and which extends well beyond the first year in immunosuppressed organ transplant recipients (Matthew, Transplant Proc 1999; 31: 1102). Finally, there are many complications from drug toxicities in these patients, including cataracts, tremors, hyperplasia of the gums and overgrowth of body hair (Almanar et al., Transplant Prac 1998; 31: 2519).

In summary, immunosuppression drugs are responsible for much of the mortality and morbidity of patients with transplanted organs, but these drugs will continue to be used because they delay the problems of chronic rejection and organ failure (Hasselder, Professional Nurse 1999; 14: 771).

Immunological diseases including those diseases considered to be autoimmune diseases such as rheumatoid arthritis, thyroid disease and diabetes are caused by the cells responsible for immunological rejection. Immunosuppression drugs are used to suppress immunological rejection reactions to non-self antigens of the grafts (Hallovan, Am J Med Sci 1997; 313: 283). If the cells responsible for immunological rejection reactions could be deleted, there would be no need to treat the patients with immunosuppression drugs. This opens a new door for the treatment of immunological diseases

B) Radiation Therapy. Chemotherapy, surgery and radiation therapy are the three major cancer treatment entities. Radiation therapy is used in over 60% of all cancer patients at some point during the course of their disease (Dunne-Daly, Semin Oncol Nurs. 1999; 15:250). It has become a significant therapy due to the growth of knowledge in radiation physics, computer tomography, etc. One of the basic problems in radiation therapy is killing only tumor cells, while sparing normal cells (Joensuu & Tenhunen, Acta Oncol 1999; 38 (Suppl 13):75). During the past two decades a wide variety of agents have been studied for their ability to deliver radiation to cancer cells. (Norman Coleman, Int. J. Radiation Oncol Biol Phys, 1998; 42:787). Such agents are called radiosensitizers. Other agents provide protection of normal tissues during radiotherapy. These are called radioprotectors.

Radiosensitizers must be at the right place at the right time in the right concentration. The problem is that many of the most effective radiosensitizers also are toxic. Cisplatin is an example of a toxic but potent radiosensitizer (Monagham, Lancet 1999; 353:1288). The lack of selective delivery systems makes it difficult to concentrate the right amount of radiosensitizer at the right place, thus posing a serious limitation to the effectiveness of radiotherapy.

C) Drug Delivery. Drug delivery systems usually involve devices (e.g., insulin pumps) or a variety of carriers designed to slowly release drugs into major anatomical compartments such as the gastrointestinal tract, genito-urinary tract or the blood circulation. For example, liposomes are a delivery system designed to release drugs into the blood circulation. The problem with such systems is that they are not designed to deliver drugs to specific cells (Schally & Nagy, Europ J Endoctinol 1999; 141:1). As a consequence, drugs are delivered indiscriminately. Because of this indiscriminate delivery, much larger doses of drugs must be used. In diseases such as cancer, the use of larger drug doses causes serious clinical problems with drug toxicities. Indeed, cancer patients often suffer more from the side effects of their treatment than from the effects of their cancer. In spite of this, contemporary thinking in cancer treatment is to use more drugs. For example, the 7^{th} Edition (1999) of the International Union Against Cancer's Manual of Clinical Oncology (page 278) states that, “considerable pre-clinical data and clinical experience support the hypothesis that maximally effective therapy is achieved if maximally tolerated or even supra-lethal doses of chemotherapy are administered.” The cost of giving maximally tolerated or supra-lethal doses of chemotherapy are immense suffering and unacceptable qualities of life. The suffering and unacceptable qualities of life are caused by drug toxicities.

The problem of drug toxicities would be solved if drug delivery systems could be developed that delivered anti-cancer drugs only to cancer cells and not to normal cells. One approach to this problem has been through the use of drug-laden antibodies that recognize and react with antigens that are present only on cancer cells. There are many such antigens, and some of them have been used in cancer treatment paradigms.

For example, transferrin receptor antigens are present on the surface of cancer cells but not on the surface of most normal cells (Whitney et al., Cancer 1995; 76: 20). Thus, cancer drugs have been coupled to anti-transferrin receptor antibodies and used to deliver drugs to patients with brain cancer (Laske et al., Neurosurg 1997; 41:1039). Similar studies have been done with isotope-labeled antibodies in patients with lymphomes (Vose et al., Leukemia & Lymphoma 2000; 38: 91). This antibody-mediated approach to drug delivery has several serious problems. For example, the antibodies are produced in laboratory animals and can cause undesirable reactions when given to another species, such as human beings (Muraszko et al., Cancer Res 1983; 53: 3752).

A more rational approach to the problem of drug delivery in cancer is to design drug carriers that also are the normal ligands for receptors on cancer cells. A ligand is a molecule that fits uniquely into a specific receptor, analogous to a key that fits uniquely into a specific lock. The requirement for this approach is that the receptor is present on the surface of cancer cells and not on the surface of normal cells. Such is the case for transferrin receptors that bind the protein ligand responsible for transportation of iron in the blood. This protein ligand has been modified to carry anti-cancer drugs, and the efficacy of this ligand-receptor drug delivery system has been shown in patients with acute leukemia (Faulk et al., Mol Biotherapy 1990; 2: 57).

Also, the drug-carrying ligand has been shown capable of carrying several different types of anti-cancer drugs (Berczi et al., Arch Biochem Biophy 1993; 300: 356, and Beyer et al., J Med Chem 1998; 41: 2701) through several different types of chemical bonds (Kratz et al., J Pharm Sci 1998; 87: 338). However, this approach is limited to the use of protein ligands involved in the transport of heavy metals, such as iron.

### SUMMARY OF THE INVENTION

An object of this invention is to achieve the targeted delivery of cytotoxic drugs to lymphocytes responsible for the rejection of transplanted tissues, such as bone marrow, kidneys or hearts.

Another object of this invention is to achieve the use of a non-toxic naturally existing delivery system to transport high concentrations of radiosensitizers to cancer cells.

Still another object of this invention is to achieve the targeted delivery of drugs in medical diagnosis and treatment of cancer.

These and other objects of this invention are achieved by this invention as described in the detailed description of the preferred embodiments set forth below.

### DETAILED DESCRIPTION OF THE PREFERRED EMBODIMENTS OF THE INVENTION

This invention provides several mechanisms for targeted delivery to targeted cells.

A) Transplantation. In one embodiment of this invention the immunological cells responsible for rejection reactions in transplant recipients are deleted. This is supported by the observation that non-self recognition reactions are mediated by T cell receptors on recipient lymphocytes and a combination of antigen and major histocompatibility complex on donor cells (Turvey & Wood, Int Surg 1999; 84: 279). When the donor and recipient cells come together, the recognition reaction is accompanied by a co-stimulator reaction that triggers cellular proliferation and up-regulation of transferrin receptors (Pattanapanyasat & Hoy, Eur J Haematol 1991; 47: 140). Evidence that transferrin receptor up-regulation associates with cellular proliferation is that the proliferation does not occur if the transferrin receptor is blocked by antibodies to transferrin receptor (Bayer et al., J Leuk Biol 1998; 64: 19). Antibodies to transferrin receptor also block the recognition reaction of recipient cells to donor cells (Bayer et al., Transplant Immunol 1999; 7: 131), and prolong the survival of transplanted hearts in genetically incompatible recipients (Woodward et al., Transplantation 1999; 68: 1369). Thus, it is clear that transferrin receptors are important in the immunological rejection reactions that transplant recipients mount to their grafts.

The immunobiology of purging T lymphocytes from the body is to initiate a pathway of programmed cell death by a process known as apoptosis (Pinkoski & Green, Cell Death & Dif 1999; 6: 1174). This can occur in the thymus gland or in the peripheral circulation (Le Bon et al., Int Immunol 1999; 11: 373). Although there are several mechanisms of apoptosis, they all function to eliminate selected cells from the body (Martinez & Kraus, Int Rev Immunol 1999; 18: 527). The present invention is a novel way to eliminate recipient immunosurveillance T lymphocytes after they have confronted donor antigens of the graft but before they proliferate to produce a clone of T lymphocytes that attack and ultimately are responsible for rejecting the graft. The strategy of this approach is to eliminate the donor specific immunosurveillance lymphocytes by clonal deletion via apoptosis, then a rejecting clone of T lymphocytes will not develop and there will be no need for immunosuppression to protect the graft. This same strategy can be applied to the elimination of autoimmune lymphocytes, such as those with specificities for thyroid, brain, joint or insulin producing cells, then an autoreactive clone of T lymphocytes will not persist and autoimmune disease will abate, again disallowing the need for immunosuppressive therapies.

The invention is a drug-protein conjugate to be used for the targeted delivery of a cytotoxic drug to immunosurveillance T lymphocytes in transplant recipients after the recipients have received their grafts but before the cells proliferate to form a clone of cells to reject the transplanted organ. Targeted delivery of drugs is possible in this invention because the protein in the drug-protein conjugate is transferrin and the receptor on the targeted cells is transferrin receptor, which is up-regulated to the surface of the patient's progenitor immunosurveillance T lymphocytes. In addition, the drug in the drug-transferrin conjugate can be methotrexate, which is known to cause apoptosis and clonal deletion of activated peripheral T lymphocytes (Genestier et al., J Clin Invest 1998; 102: 322). Other than being present on the surfaces of cancer cells (Yeh et al., Vox Sang 1984; 46: 217), infected cells (Ohno et al., Virology 1997; 238: 305) and antigen-stimulated T lymphocytes (Bayer et al., J Leuk biol 1998; 64: 19), transferrin receptors usually are not present on the surface of normal, adult, resting cells (Berczi et al., Arch Biochem Biophy 1993; 300: 356). Thus, cells in the transplanted organ will not be affected, and the only recipient cells to be eliminated by the methotrexate-transferrin conjugate will be the immunosurveillance T lymphocytes before they have had an opportunity to expand into a clone of rejecting cells.

The way to realize this invention of targeted drug delivery to prolong graft survival without the use of immunosuppression drugs is to focus on the use of transferrin, which carries iron in the blood. Transferrin can be obtained by isolation from blood plasma, from commercial suppliers, or from recombinant technology (Ali et al., J Biol Chem 1999; 274: 24066). To form the drug-protein conjugate, the transferrin molecule must be modified in such a way as to prepare it to be coupled with a cytostatic drug. The drug can be an apoptosis initiator such as methotrexate, a cytotoxic antibiotic such as adriamycin or an alkylating agent such as taxol, but any compound toxic to cells can be used, including plant toxins such as ricin and bacterial mutant toxins such as modified diptheria toxin (Laske et al., Nature Med 1997; 41: 1039). Several coupling processes such as glutaraldehyde coupling (Berczi et al., Arch Biochem Biophys 1993, 300:356), disulfide coupling (Sasaki et al., Jap J Cancer Res, 1993; 84:191) and benzolyl hydrazone coupling (Kratz et al., J Pharm Sci 1998; 87:338) have been used to couple transferrin with other compounds and can be used in embodiments of this invention. DTPA (diethylene triamine penta acetate) coupling can also be used as bridge to couple contain isotopes to transferrin for purposes of tumor imaging. The broad variety of coupling procedures allows the conjugation of a wide range of cytotoxic drugs to transferrin, resulting in either permanent or dissociable bonding of cytotoxic drugs with the transferrin molecule. Following the coupling reaction, drug-protein conjugates can be separated from uncoupled drug and free protein, preferably by using chromatographic or dialysis procedures.

Technical details of the conjugation procedure can vary, but the requirement of any procedure is to prepare defined conjugates that are active in binding and killing experiments with cancer cells, and that do not bind or kill significant numbers of normal cells. In light of these requirements, a suitable procedure is to mix one milliliter of transferrin (0.5 mM) with one milliliter of adriamycin (8.5 mM) in 150 mM sodium chloride for 4 minutes, and then add one milliliter of 21.5 mM glutaraldehyde in 150 mM sodium chloride and mix 4 minutes. The preceding reaction is a coupling procedure, which is stopped by the addition of 0.8 milliliters of 37.2 mM ethanolamine in 150 mM sodium chloride and 10 mM Hepes buffer (pH8) and vortexed for 4 minutes. The mixture (3.8 milliliters) then is transferred to dialysis tubing (molecular weight cutoff of 12,000-14,000) and dialyzed against 0.5 liters of hepes-buffered saline in the dark at 5 C for 3 hours. The dialysis should be repeated at least once with fresh Hepes-buffered saline. The mixture then is centrifuged at 1600 g for 10-minutes at 4° C. and the supernatant is chromatographed at a flow rate of 22 milliliters per hour on a 2.6×34 cm column of sepharose CL-4B, previously equilibrated in Hepes-buffered saline and calibrated at 5° C. with blue dextran, transferrin and cytochrome C. Elution from the column is monitored at 280 mm, and 3.8 milliliter fractions are collected. The concentrations of transferrin and adriamycin in each fraction is calculated by successive approximation from standard curves for transferrin and adriamycin, determined by using 280 nm for transferrin and 295 nm for adriamycin. With minor modifications, this coupling procedure can be used to prepare transferrin conjugates of other cytotoxic drugs, such as transferrin conjugates of methotrexate or transferrin conjugates of immunosuppressive drugs such as cyclosporin.

When pure drug-protein conjugates are isolated, they should be characterized by polyacrylamide gel electrophosesis to determine their molecular weight, and the number of drug molecules per protein molecule should be determined. Experience with drug-protein conjugates in other systems has shown that a functional drug:protein ratio is 2-3 molecules of drug per molecule of protein (Berczi et al., Arch Biochem Biophy 1993; 300: 356). However, with this invention, ratios of less than one molecule of drug per molecule of protein can be effective. The next preferable two steps in carrying out this invention are to (a) determine if the conjugate binds to receptors on the surface of activated T lymphocytes and not to resting T lymphocytes, and (b) determine if the conjugate kills activated T lymphocytes and not resting T lymphocytes. The binding studies can be done by using flow cytometry, and the killing studies can be done by using microculture techniques to determine the concentration of free drug required to kill 50% of a culture of activated T lymphocytes compared to the concentration of drug in the drug-protein conjugate required to kill the same number of activated T lymphocytes. Experience with drug-protein conjugates in other systems indicates that approximately 10-fold more free drug compared to the drug in drug-protein conjugates should be required to kill the same number of activated T lymphocytes. However, with this invention, a twenty-fold or even one-hundred fold less drug in drug-protein conjugate compared to free drug alone can be effective. For the conjugate to be efficacious, it also should kill only a minimum of unactivated lymphocytes.

While the present invention has been described in relation to transferrin being the delivery protein, it is known that other proteins exist in the body which are capable of binding to receptor sites on cells. If the receptor site is activated in tumor cells, and is inactive in resting normal cells, then any protein or other compound which binds to such a receptor site can be used to deliver the cytostatic drugs of the present invention. One example of such other protein are the transcobalamines, which deliver vitamins, especially vitamin B12, to cells in the human body. Also, drug conjugates of the vitamin folic acid have been used in this capacity (Reddy & Low, Crit. Rev Ther Drug Car Sys. 1998; 15; 587).

After the drug-protein conjugate has been prepared, purified, characterized and validated for cellular binding and killing properties, and, when the binding and killing experiments show that the conjugate binds to and kills activated T lymphocytes but not resting or unactivated lymphocytes, the conjugate then will be aliquoted and sterilized for animal studies. The sterilization process can be done by exposure to irradiation, such as by using a cesium irradiator, or it can be done by using millipore filtration techniques. When sterile conjugates are available, in the first instance they will be used in animal studies to determine pharmacokinetic and safety parameters to be employed in the design of clinical studies

The method of preparation for the first drug-protein conjugate designed to initiate clonal deletion of activated T lymphocytes in transplant recipients has been described in the preceding paragraphs. The general nature is to test the first drug-protein conjugate designed for targeted delivery of cytotoxic drugs to the cells that are specifically responsible for organ rejection, and the purpose is to initiate clonal deletion of activated T lymphocytes to obviate the need for prolonged immunosuppression in patients. The benefit to science and/or society is the information that transplantation can be done without the prolonged use of damaging and toxic immunosuppression drugs, and that a more acceptable quality of life can be maintained after transplantation. The risk is low (e.g., no toxicity) and the possible benefit is high (e.g., retention of a functional transplant without the use of prolonged immunosuppression), thus the risk/benefit ratio is very favorable. Thus, embodiments of this invention describe the first targeted delivery of cytotoxic drugs carried to activated T lymphocytes in patients with transplanted organs. The goal of this approach is to eliminate the present need for long-term immunosuppression.

B) Radiation Therapy. Other embodiments of this invention concern the use of a non-toxic naturally existing delivery system to transport high concentrations of radiosensitizers to cancer cells. These embodiments of this invention also can use transferrin, which is a glycoprotein that circulates in the blood and delivers iron to cells which express transferrin receptors on their surface (Richardson & Ponka, Biochim Biophys Acta 1997. 1331:1). Transferrin receptor is a proliferation marker which is continuously expressed on the surface of cancer cells (Faulk et al., Lancet 1980; 2:390: Kemp, Histol & Histopathol 1997; 12:291). As described above for the earlier embodiments of this invention, tumor cells express large numbers (10⁴-10⁶/cell) of transferrin receptors on their surface, while normal cells rarely express these receptors on their surface (Klausner et al., Cell 1993; 72:19).

The use of transferrin as a carrier for the targeted delivery of radiosensitizers makes it possible to deliver high concentrations of radiosensitizers substantially only to cancer cells, thus allowing the use of lower total doses of radiosensitizers which is important in the case of toxic sensitizers such as cisplatin. The use of targeted delivery for radiosensitizers also diminishes collateral damage to normal cells, thereby sparing normal cells and allowing normal tissues to continue their physiological functions. The net effect of this invention is to improve the outcome of radiotherapy and to decrease toxic side effects. This will diminish side-effects and improve the quality of life for cancer patients.

There is a wide spectrum of radiosensitizers that act on different cell processes such as angiogenesis, signal transduction, apoptosis, etc. The reason for this variety of radiosensitizers is that radiation damage is tissue specific (Withers & McBride in Principles and Practice of Radiation Oncology (ed 3), p. 79, 1998). In addition, the biology of cancer cells is altered and this often results in the development of resistance to radiation and other types of drug therapies. However, sensitive and resistant cells express large numbers of transferrin receptors on their surfaces (Barabas & Faulk, Biochem Biophys Res Com 1993; 197:702). Since binding of transferrin to its receptor triggers endocytosis of the ligand-receptor complex, transferrin is a perfect molecule to deliver compounds into cells that have transferrin receptors on their surfaces (Hein et al., Euro J Cardio-Thor Surg 1998; 13:460; Broodwell et al., Exper Neurol 1996; 142:47).

As transferrin is an excellent carrier for delivering compounds into drug sensitive and drug resistant cancer cells, the preferred targeted delivery system in this invention consists of transferrin coupled to radiosensitizers. Transferrin for use in human patients is commercially available (for example, from the Finnish Red Cross). Several coupling processes such as glutaraldehyde coupling (Berczi et al., Arch Biochem. Biophys 1993, 300:356), disulfide coupling (Sasaki et al., Jap J Cancer Res 1993; 84:191) and benzolyl hydrazone coupling (Kratz et al., J Pharm Sci 1998; 87:338) have been used to couple transferrin with other compounds. The broad variety of coupling procedures allows the conjugation of a wide range of radiosensitizers to transferrin, resulting in either permanent or dissociable bonding of radiosensitizers with the transferrin molecule. Following the coupling reaction, after intensive dialysis, the conjugates should be separated from the uncoupled components by chromatographic or dialysis procedures. Cisplatin is a preferred radiosensitizer, although any known radiosensitizer may be substituted therefor.

After conjugates of transferrin with a radiosensitizer are prepared, they can be characterized for molecular weight, as for instance by using polyacrylamide gel electrophoresis followed by other methods, depending on the physiochemical characteristics of the radiosensitizer used to prepare the transferrin conjugate. This can be followed by binding studies using flow cytometry to determine the ability of the conjugates to bind cancer and normal cells. Only conjugates that bind to cancer cells and not to normal cells are preferably selected for radiotoxicity assays using drug sensitive and drug resistant cancer cells.

The radiotoxicity assay will utilize the MTT tetrazolium colorimetric assay (Vistica et al., Cancer Res 1994; 51:2515), which will indicate the most potent transferrin-radiosensitizer ratio and the optimum concentration of conjugate for maximal radiosensitization of drug sensitive and drug resistant cells. Conjugates selected from the cell bonding and killing studies can be used in animal studies to determine pharmacokinetic and safety parameters before being employed in the design of clinical studies. This can be done as xenografted human tumor cells in nude mice. The aim of this invention is to introduce the concept of delivering radiosensitizers to only cancer cells, which will provide substantial improvement in the outcome of radiotherapy.

The conjugates of transferrin with a radiosensitizer can be used in patients with a defined stage of an accurately diagnosed cancer. The transferrin-radiosensitizer conjugate can be administered intravenously in a dose that can be 10-fold less and preferably 20-fold or even 100-fold less than that which would be used if only free radiosensitizer was given. After administration of the conjugate, the patient can be administered radiotherapy, using conventional amounts of radiation. Biochemical, cellular and radiological parameters of patients and controls can be analyzed following radiotherapy to determine the efficacy of the invention.

While the present invention has been described in terms of transferrin as the carrier for targeted delivery of the radiosensitizers, it will be appreciated that other proteins or compounds may be used, such as any other protein (e.g., transcobalamin) or other compound that is capable of specific binding to receptors on cancer cells. The receptors should be of a nature such that they are activated in tumor cells and are substantially inactive in normal resting cells. The radiosensitizers should retain their radiosensitizing properties when conjugated, or when delivered to the cancer cells, and the transferrin or other protein or other compound should retain its ability to bind to the above-described cellular receptors therefor when in the conjugate.

The risk is much less using a carrier system than using the radiosensitizer by itself. The risk benefit ratio is very favorable. The lower concentration of radiosensitizer administrated will result in higher local concentrations of the radiosensitizer in cancer cells, while normal cells will be spared. This distribution will increase the effectiveness of radiotherapy. The transferrin radiosensitizer conjugate will help to determine the location and size of tumors, as well as sensitizing them to radiation. The invention does not use antibodies, thus there are not the problems which are present with antibody delivery systems, such as anaphylactic reactions, patch-cap-secretion reactions and antigenic drift. By using the transferrin targeted delivery systems, it is possible to administer lower doses of toxic radiosensitizers, which decrease toxic side-effects. Thus, this invention will improve the quality of life and lifetime of cancer patients.

C) Drug Delivery. This embodiment of the invention concerns the targeted delivery of drugs in medical diagnosis and treatment of cancer. This aspect of the invention is a new approach to the targeted delivery of drugs. This approach involves blood proteins that normally carry vitamins such as folic acid (Reddy & Low, Crit. Rev Ther Drug Cat Sys 1998; 15:587), and vitamin B12 (Seetharam, Vitamins & Hormones 2000; 59: 337). The invention concerns modifying transcobolamin receptors on cancer cells.

Transcobolamins deliver vitamin B12 (cobalamin) to cells with transcobalamin receptors on their surfaces, where vitamin B12 is transferred to the cells via receptor-mediated endocytosis and utilized to convert homocysteine to methionine (Seetharam, Annu Rev Nutr 1999; 19: 173). The biologically active receptor functions as a noncovalent dimer of 124 kDa in cell membranes (Seetharam et al., J Nutr 1999; 129: 1761), and antibodies to the receptor block cellular proliferation (McLean et al., Leukemia & Lymphoma 1998; 30: 101).

Experimental studies have shown that the regulation of transcobalamin receptor expression in cell membranes correlates with cellular proliferation, in which they increase, and with cellular differentiation, in which they decrease (Amagasaki et al., Blood 1990; 76: 1380). Transcobalamin receptors are characteristic of cancer cells (Collins & Hogenkamp, J Nucl Med 1997; 38: 717, and Fiskerstrand, J Biol Chem 1998; 273: 20180). Thus, the coupling of transcobalamins with anti-cancer drugs will provide a targeted delivery system for drugs.

The use of targeted delivery in cancer is not limited to vitamin B12, for a similar receptor mechanism has been reported for folic acid (Holm et al., Arch Biochem Biophy 1999; 366: 183, and Triplett & Bertino, J Chemotherapy 1999; 11: 3). As a matter of fact, other than transferrin, which has already been described by the inventor for the carrying of anti-cancer drugs, any protein or other substance which binds to receptors on cell surfaces, which receptors are activated in tumor cells and are inert or relatively inactive in normal cells, can be utilized via a conjugation procedure to deliver any tumor drug to tumor cells. The other protein or other compound should of course be pharmacologically acceptable in the human body, and should maintain its ability to bind to such receptors even after conjugation.

Of course, the anti-cancer agent or drug which is so delivered to the tumor cells must retain its ability to kill such cells. The preferred anti-cancer drugs are the drugs which have been approved for the treatment of tumors. In addition to the adriamycin and taxol mentioned above, such drugs can include such diverse compounds as cysplatin and methotrexate.

A number of techniques may be used to couple transcobalamin to anti-cancer drugs. Several coupling processes such as glutanaldehyde coupling (Berczi et al., Arch Biochem. Biophys 1993, 300:356), disulfide coupling (Sasaki et al., Jap J Cancer Res 1993; 84:191) and benzolyl hydrazone coupling (Kratz et al., J Pharm Sci 1998; 87:338) have been used to couple transferrin with other compounds. Similar coupling processes can be used to produce the conjugates of the present invention. The broad variety of coupling procedures allows the conjugation of a wide range of antitumor drugs to transcobalamin or other transport agents. Following the coupling reaction, after intensive dialysis the conjugates should be separated from the coupled components by chromatographic or dialysis procedures.

The use of vitamin-carrying ligands coupled to anti-cancer drugs will allow treatment protocols with lower drug doses, and the combined effects of sparing normal cells and using smaller amounts of drugs will cause less drug toxicities and improve the quality of life for cancer patients. Normal cells and tissues are spared cytotoxic damage because any receptors that might be on their cell surfaces can be down-regulated by the administration of exogenous vitamin B12, which has more of an effect on normal than on cancer cells. There also are data suggesting that the transcobalamin receptor is regulated by cortisone (Bose et al., Biochem J 1995; 310: 923), offering possibly another approach to the control of the presentation of transcobalamin receptors by normal cells.

The best way to realize this invention of targeted drug delivery with vitamin-carrying protein ligands is to focus on the use of transcobalamin II, which carries vitamin B12, or cobalamin. Transcobalamins can be obtained by isolation from blood plasma, from commercial suppliers, or from recombinant technology (McLean et al., Blood 1997; 89: 235). The transcobalamin molecule must be modified in such a way as to prepare it to be coupled with an anti-cancer drug. The anti-cancer drug can be a cytotoxic antibiotic such as adriamycin or an alkylating agent such as taxol, but any compound which is toxic to cells can be used, including plant toxins such as ricin and bacterial mutant toxins such as modified diphtheria toxin (Laske et al., Nature Med 1997; 41: 1039). Following the coupling reaction, drug-protein conjugates can be separated from uncoupled drug and free protein, preferably by chromatographic or dialysis procedures.

When pure drug-protein conjugates are isolated, they should be characterized by polyacrylamide gel electrophosesis to determine their molecular weight, and the number of drug molecules per protein molecule should be determined. Experience with drug-protein conjugates in other systems has shown that a functional drug:protein ratio is 2-3 molecules of drug per molecule of protein (Berczi et al., Arch Biochem Biophy 1993; 300: 356). However, a protein ratio of less than 1 molecule of drug per molecule of protein can be used in embodiments of this invention.

The next preferred two steps in carrying out this invention are to (a) determine if the conjugate binds to receptors on the surface of cancer cells and not to normal cells, and (b) determine if the conjugate kills cancer cells and not normal cells. The binding studies can be done by using flow cytometry, and the killing studies can be done by using microculture techniques to determine the concentration of free drug required to kill 50% of a culture of cancer cells compared to the concentration of drug in the drug-protein conjugate required to kill the same number of cancer cells. Experience with drug-protein conjugates in other systems indicates that approximately 10-fold more free drug compared to the drug in drug-protein conjugates is required to kill the same number of cancer cells. In embodiments of this invention, a 20 or even 100-fold ratio can be used. For a conjugate to be efficacious, it also should kill only a minimum of normal cells.

Technical details of the conjugation procedure can vary, but the requirements of any procedure is to prepare defined conjugates that are active in binding and killing experiments with cancer cells, and that do not bind or kill significant numbers of normal cells. In light of these requirements, a suitable procedure is to mix one milliliter of transcobalamin II (0.5 mM) with one milliliter of adriamycin (8.5 mM) in 150 mM sodium chloride for 4 minutes, and then to add one milliliter of 21.5 mM glutaraldehyde in 150 mM sodium chloride and mix 4 minutes. The preceding reaction is a coupling procedure, which is stopped by the addition of 0.8 milliliters of 37.2 mM ethanolamine in 150 mM sodium chloride and 10 mM Hepes buffer (pH 8) and vortexed for 4 minutes. The mixture (3.8 milliliters) then is transferred to dialysis tubing (molecule weight cutoff of 12,000 B 14,000) and dialyzed against 0.5 liters of Hepes-buffered saline in the dark at 5 C for 3 hours. The dialysis can be repeated once with fresh Hepes-buffered saline. The mixture can then be centrifuged at 1600 g for 10 minutes at 4° C. and the supernatant is chromatographed at a flow rate of 22 milliliters per hour on a 2.6×34 cm column of sepharose CL4B, previously equilibrated in Hepes-buffered saline and calibrated at 5° C. with blue Dextran, transcobalamin II and cytochrome C.

Elution from the column can be monitored at 280 mM, and 3.8 milliliter fractions can be collected. The concentrations of transcobalamin II and adriamycin in each fraction can be calculated by successive approximation from standard curves for transcobalmin and adriamycin. Fractions collected from the sepharose CL-4B column then can be analyzed by sodium dodecyl sulfate-polyacrylamide gel electrophoresis. With only minor modifications, this coupling procedure can be used to prepare transcobolamin conjugates of other cancer drugs, and the procedure also can be used to prepare anti-cancer conjugates of radiolabeled transcobalamin for use in tumor imaging, cancer diagnosis and the monitoring of cancer mass during treatment. For example, qualitative and quantitative tumor imaging can be accomplished by means of DTPA-chelated isotopes bound to the carrier protein, which in this case is transcobalamin.

After the drug-protein conjugate has been prepared, purified, characterized and validated for cellular binding and killing properties, and when the binding and killing experiments show that the conjugate binds to and kills cancer cells but not normal cells, then the conjugate can be aliquoted and sterilized for animal studies. The sterilization process can be done by exposure to irradiation, such as by using a cesium irradiator (Yck & Faulk, Clin-Immunol Immunopatholy 1987; 32:1, or it can be done by using millipore filtration techniques. When sterile conjugates are available, in the first instance they can be used in animal studies to determine pharmacokinetic and safety parameters to be employed in the design of clinical studies.

The aim of all this work is to introduce the concept of targeted delivery of drugs into cancer treatment protocols, and the goal of this work is to eliminate drug toxicities through targeted delivery, and to improve the quality of life for patients with cancer. The method of preparation for the first drug-protein conjugate composed of a vitamin-carrying blood protein and an anti-cancer drug has been described in the preceding paragraphs. The general nature is to test the first drug-protein conjugate composed of a vitamin-carrying blood protein and an anti-cancer drug, and the purpose is to develop the concept of targeted delivery of drugs to cancer patients. The benefit to be gained is that the patient's cancer will be treated without adding drug toxicities and with the possibility of decreasing the mass of the cancer. The cancer can be treated without or with less collateral damage, and a more acceptable quality of life of the cancer patient can be maintained during and after treatment. The risk/benefit ratio with this invention is low (e.g., no toxicity) and the possible benefit is high (e.g., remission), thus the risk/benefit ratio is very favorable.

The drug-protein conjugate can be administered intravenously in a dose that will be 10-fold less (and preferably 20-fold or even 100-fold less) than that used for the free drug. In summary, the invention involves the first study of targeted delivery of an anti-cancer drug carried to cancer cells by a normal vitamin-carrying blood protein.

An advantage of this invention is that the various embodiments can be utilized together to take advantage of a combined therapy, which has the power to apply in all three cases (i.e., transplantation, radiosensitizers, and the use of transcobalamin). The strategy here is a process of combinations and permutations. For example, in transplantation, transferrin conjugates of a cytotoxic drug such as adriamycin can be used to kill T cells, while transcobalamin conjugates of an immunosuppressive drug such as cyclosporin can be simultaneously used in the same patient to selectively suppress the immune system. In this way, only the immunologically activated, or allogeneically activated cells, that is, those which are activated as a result of the graft, are targeted by virtue of their up-regulated transferrin receptors. Those cells can be treated with both cytotoxic and immunosuppressing drugs which attack through both the transferrin receptor and the transcobalamin receptor.

In addition, one of the carriers (either transferrin or transcobalamin) can be labeled with a cytotoxic drug and the other with an isotope, in order to either kill and image simultaneously, or to kill simultaneously with a cytotoxic drug (e.g., adriamycin) on the one hand and a high energy isotope on the other.

Thus, the combined therapy that targets the same cells through two different receptors (i.e., transferrin receptor and transcobalamin receptor). This can be mathematically expressed as follows:

Cytotoxicity (drug A)/Cytotoxicity (drug B)

Cytotoxicity (drug C)/Cytotoxicity (isotope D)

Cytotoxicity (drug E)/Imaging (isotope F)

The above model can be expanded by:

Transferrin-conjugate A/Transferrin-conjugate B

Transferrin-conjugate C/Transcobalamin-conjugate D

Transcobalamin-conjugate E/Transcobalamin-conjugate F

When a cell is metabolically disturbed by activation, infection, cancer, etc., its response is to up-regulate certain receptors (e.g., transferrin receptor, transcobalamin receptor, etc.) and that in so doing it becomes a target for the specific ligand (e.g., transferrin, transcobalamin, etc.) which can be labeled with different molecules (e.g., adriamycin) or atoms (e.g., isotopes) for purposes of treatment (e.g., cancer, infectious disease, transplantation, autoimmunity, etc.) or imaging (e.g., cancer).

What is claimed is:

1. The use of a conjugate of a binding moiety which binds to a specific receptor on activated T-lymphocytes responsible for the rejection of a transplanted organ or transplanted bone marrow and a cytotoxic drug for targeted delivery of the cytotoxic drug to the lymphocytes responsible for the rejection of the transplanted organ or transplanted bone marrow.

2. The use of claim 1 wherein the binding moiety is transferrin or transcobalamin.

3. The use of claim 1 wherein the cytotoxic drug is selected from the group consisting of: methotrexate, an antibiotic, adriamycin, an alkylating agent, taxol, a plant toxin, ricin, a bacterial mutant toxin, modified diptheria toxin, an immunosuppressive drug, and cyclosporin.

4. The use of claim 1 wherein the transplanted organ is a kidney or a heart.

5. The use of claim 1 wherein the transferrin is conjugated to the cytotoxic drug by glutaraldehyde coupling, disulfide coupling, benzolyl hydrazone coupling or DTPA coupling.

6. A method of transplanting an organ or a tissue into a patient, comprising:
transplanting the organ or tissue into the patient;
administering to the patient a conjugate of a) a binding moiety which binds to a specific receptor on lymphocytes that would, in the absence of the conjugate, be responsible for rejection of the transplanted organ, and b) a cytotoxic drug, wherein the conjugate is administered in an amount effective to prevent rejection of the transplanted organ by the patient.

7. The method of claim 6 wherein the binding moiety is transferrin or transcobalamin.

8. The method of claim 6 wherein the cytotoxic drug is selected from the group consisting of: methotrexate, an antibiotic, adriamycin, an alkylating agent, taxol, a plant toxin, ricin, a bacterial mutant toxin, modified diptheria toxin, an immunosuppressive drug, and cyclosporin.

9. The method of claim 6 wherein the transplanted organ or tissue is a kidney or a heart or bone marrow cells.

10. The method of claim 6 wherein the transferrin is conjugated to the cytotoxic drug by glutaraldehyde coupling, disulfide coupling, benzolyl hydrazone coupling or DTPA coupling.

11. The use, for transport of high concentrations of radiation sensitizers, to cancer cells of a conjugate with a radiation sensitizer of a binding moiety that binds to a specific receptor on cancer cells.

12. The use of claim 11 wherein the binding moiety is transferrin.

13. The use of claim 11 wherein the radiation sensitizer is cisplatin.

14. The use of claim 11 wherein the transferrin is conjugated to the cytotoxic drug by glutaraldehyde coupling, disulfide coupling, benzolyl hydrazone coupling or by DTPA coupling.

15. The use of claim 14 wherein the transferrin is conjugated to a cytotoxic agent or isotope by DTPA coupling.

16. A method of transporting high concentrations of radiosensitizers to cancer cells in a patient, comprising:
administering to the patient a conjugate of a binding moiety which binds to a specific receptor on cancer cells with a radiation sensitizer.

17. The method of claim 16 wherein the binding moiety is transferrin.

18. The method of claim 16 wherein the radiation sensitizer is cisplatin or adriamycin.

19. The method of claim 16 wherein the transferrin is conjugated to the cytotoxic drug by glutaraldehyde coupling, disulfide coupling, benzolyl hydrazone coupling or DTPA for isotope chelation.

20. The method of claim 16 wherein the transferrin is conjugated to a cytotoxic drug or isotope by DTPA coupling.

21. The use, for the targeted delivery of an anti-cancer drug to cancer cells for medical diagnosis and/or treatment of cancer, of a conjugate of a vitamin carrying binding moiety that binds to a specific receptor on cells in a patient and an anti-cancer drug.

22. The use of claim 21 wherein the binding moiety is a transcobalamin.

23. The use of claim 21 wherein the anti-cancer drug is selected from a group consisting of: adriamycin, taxol, cisplatin, methotrexate, a plant toxin, ricin, a bacterial mutant toxin, and a modified diptheria toxin.

24. The use of claim 22 wherein the transcobalamin is transobalamin II.

25. The use of claim 21 wherein the transcobalamin is conjugated to the anti-cancer drug by glutaraldehyde coupling, disulfide coupling, benzolyl hydrazone coupling or DTPA coupling.

26. A method of medical diagnosis and/or treatment of cancer in a patient, comprising:
administering to the patient a conjugate of a binding moiety which binds to a specific receptor on cancer cells and an anti-cancer drug or isotope, wherein the conjugate is administered in an amount effective to treat and/or diagnose the cancer in the patient.

27. The method of claim 26 wherein the binding moiety is transcobalamin.

28. The method of claim 26 wherein the anti-cancer drug is selected from the group consisting of: adriamycin, taxol, cisplatin, methotrexate, a plant toxin, ricin, a bacterial mutant toxin, and a modified diptheria toxin.

29. The method of claim 26 wherein the transcobalamin is transobalamin II.

30. The method of claim 26 wherein the transcobalamin is conjugated to the anti-cancer drug by glutaraldehyde coupling, disulfide coupling, benzolyl hydrazone coupling or DTPA coupling.

31. A composition, comprising
a) a first conjugate of a first binding moiety which binds to a specific receptor on cancer cells on T-lymphocytes responsible for the rejection of a transplanted organ or transplanted bone marrow and a first cytotoxic drug or radiation sensitizer or anti-cancer drug; combined with
b) a second conjugate of a second binding moiety which binds to another specific receptor on cancer cells or T-lymphocytes responsible for the rejection of a transplanted organ or transplanted bone marrow and a second cytotoxic drug or radiation sensitizer or anti-cancer drug.

32. The composition of claim 31 wherein the first binding moiety is transferrin and the second binding moiety is transcobalamin.

33. A method of treating a patient with an autoimmune disease, comprising:
administering to the patient a conjugate of a) a binding moiety which binds to a specific receptor on lymphocytes that would, in the absence of the conjugate, be responsible for rejection for autoimmunity, and b) a cytotoxic drug, wherein the conjugate is administered in an amount effective to treat the autoimmune disease in the patient.
